(19) United States
(12) Patent Application Publication
(10) Pub. No.: US 2006/0292669 A1
(43) Pub. Date: Dec. 28, 2006

(54) Mammalian prickle gene

(75) Inventors: Masakazu Takeuchi, Kyoto-shi (JP); Keiko Sato, Kyoto-shi (JP); Yoshimasa Sakamoto, Kyoto-shi (JP); Maki Tawarada, Kyoto-shi (JP); Toshihisa Ohtsuka, Kyoto-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eisai Co., Ltd., Tokyo (JP)

(21) Appl. No.: 10/536,586

(22) PCT Filed: Nov. 28, 2003
(86) PCT No.: PCT/JP03/15223

§ 371 Date: Mar. 17, 2006

(30) Foreign Application Priority Data
Nov. 28, 2002 (JP) 2002-346447

(51) Int. Cl. C07K 14/705; C07K 16/28; C07H 21/04; C12P 21/06

(52) U.S. Cl. 435/69.1; 435/320.1; 435/325; 530/350; 530/388.22; 536/23.5

(57) ABSTRACT

The present invention provides the mPrickle gene which encodes a protein present in mammalian PSD fractions. The mPrickle protein is localized in synapses, and binds to the scaffold protein PSD-95. Precipitation of endogenous mPrickle using an anti-mPrickle antibody results in the coprecipitation of NMDA receptors, and thus, mPrickle can be used in drug delivery systems that target NMDA receptors. NMDA receptors are closely related to learning and memory, and are also suggested to be involved in mental disorders. Thus, in the future, mPrickle is expected to be applicable to the diagnosis and/or treatment of neurodegenerative diseases associated with learning/memory, such as mental deterioration and dementia.

### TECHNICAL FIELD

The present invention relates to a novel gene that encodes the mammalian prickle (mPrickle) protein expressed in postsynaptic densities (PSDs). The present invention also relates to methods of using said gene for producing polypeptides and fragments thereof, and antibodies against these polypeptides.

### BACKGROUND ART

“Neuronal synapses” refer to morphologically asymmetric junctions between neurons, and are central to neurotransmission. They actively modify their adhesion according to neural activity, and modulate the efficiency of neurotransmission at presynaptic and postsynaptic membranes. A postsynaptic density (PSD) is a specialized region that lines postsynaptic membranes at excitatory synapses, and where scaffolding molecules such as PSD-95 as well as neurotransmitter receptors and ion channels are present in large numbers. PSDs are thought to be deeply involved in functions of the nervous system, such as morphological changes of neurons and neuronal plasticity (Y. Yoshimura and T. Yamauchi (1997) J. Biol. Chem. 272: 26354; S. Stack et al. (1997) J. Biol. Chem. 272: 13467; Siekevitz et al. (1985) Proc. Natl. Acad. Sci. USA 82: 3494-8; Walch and Kuruc (1992) J. Neurochem. 59: 667-8). In particular, clustering and localization of membrane proteins within neurons is important for neuronal development and synapse formation. Therefore, proteins that interact with such membrane proteins are thought to influence spatial cellular distribution of membrane proteins, regulation of synaptic activities, and modulation of neurotransmitter receptor functions.

### DISCLOSURE OF THE INVENTION

“Prickle” is a protein involved in Drosophila planar cell polarity, and is known to regulate the direction of wing hair in Drosophila (herein below, Drosophila Prickle protein is referred to as “D-Prickle”) (Gubb et al. (1999) “The balance between isoforms of the prickle LIM domain protein is critical for planar polarity in Drosophila imaginal disc”, Genes Dev. 13: 2315-27). D-prickle binds to disheveled (Dsh), and is thought to inhibit fizzled (fz) signal transduction by altering the localization of Dsh. JNK (c-Jun N-terminal kinase) signaling is related to planar cell polarity signaling and as in the case of Drosophila, mammalian Dsh activates JNK signaling. These facts suggest that the Prickle protein may participate in the formation of synaptic polarity and/or JNK signaling through its interaction with Dsh in mammals as well. However, homologues in higher animals that correspond to Drosophila prickle (referred to as D-Prickle herein below) have not been isolated. Thus, an objective of the present invention is to identify and provide a D-Prickle homologue in higher animals.

The present inventors isolated MS733 as a protein concentrated in a rat brain PSD fraction. The inventors determined the protein's primary structure and performed functional analyses. The results revealed MS733 as a protein that consists of 847 amino acids, with three LIM domains at the N terminus and one PET domain. Furthermore, database search results showed that MS733 is highly homologous to the D-Prickle protein. Thus, the inventors named the MS733 protein of this invention, “mammalian Prickle” (mPrickle). Herein below, the rat-derived protein that comprises the amino acid sequence described in SEQ ID NO: 1 will be specifically referred to as “R-Prickle”. Analyses using an antibody against the mPrickle produced in this invention revealed that mPrickle is concentrated in the PSD fraction and is tightly bound to cytoskeleton. Furthermore, analyses using primary rat hippocampal neurons showed that the mPrickle localization is consistent with those of synaptophysin, PSD-95, GAD, and such. These results showed that mPrickle is a novel synaptic protein.

Further research showed that the precipitation of endogenous mPrickle using an antibody against mPrickle results in coprecipitation of NMDA neurotransmitter receptors. NMDA receptors, which are implicated in learning and memory, are known to play an important role in mental disorders and so on (J. Z. Tsien (1999) “Genetic enhancement of learning and memory in mice”, Nature 401: 63-9). Furthermore, the present inventors showed that mPrickle binds to PSD-95, which is a PSD protein. PSD-95, which is considered a model for synapse maturation, learning, and memory at the cellular level, has been shown to play an important role in the expression of long term potentiation (LTP) (El-Husseini et al. (2000) “PSD-95 involvement in maturation of excitatory synapses”, Science 290: 1364-8; Migaud et al. (1998) “Enhanced long-term potentiation and impaired learning in mice with mutant postsynaptic density-95 protein”, Nature 396: 433-9). Therefore, by making the mPrickle of the present invention a target for drug delivery, molecules closely associated with memory, such as PSD-95 and NMDA receptors, can be indirectly targeted.

More specifically, the present invention relates to:

[1] A polynucleotide encoding mammalian Prickle protein, wherein the polynucleotide comprises a sequence selected from the following nucleic acid sequences of (1) to (4):

(1) a nucleic acid sequence that encodes the amino acid sequence of SEQ ID NO: 1, or a complementary sequence thereof;
(2) the nucleic acid sequence of SEQ ID NO: 2, or a complementary sequence thereof;
(3) a nucleic acid sequence that encodes an amino acid sequence with one or more amino acid deletions, insertions, substitutions, or additions to the amino acid sequence of SEQ ID NO: 1, or a sequence complementary to said nucleic acid sequence; and
(4) a nucleic acid sequence that hybridizes with the sequence of (2) under stringent conditions.
   [2] A vector comprising the polynucleotide of [1].
   [3] A host cell comprising the polynucleotide of [1] or the vector of [2].
   [4] A method for producing a mammalian Prickle protein encoded by the polynucleotide of [1], wherein the method comprises the step of translating said polynucleotide.
   [5] A fragment of a polypeptide encoded by the polynucleotide of [1], wherein the fragment comprises at least eight amino acid residues.
   [6] An antibody directed against a polypeptide encoded by the polynucleotide of [1], or the polypeptide fragment of [5].
   [7] A nucleotide chain that encodes the polypeptide fragment of [5].
   <Polynucleotides>

Polynucleotides of the present invention can be used to express the mPrickle protein by genetic engineering. The present invention confirmed that mPrickle protein is localized in synapses, and therefore the protein can be used as a synaptic marker. In other words, polynucleotides encoding the mPrickle protein of this invention, or specific fragments thereof, can be used to detect synapses by detecting the mPrickle gene expression. Therefore, the polynucleotides of this invention can be used as reagents for synapse detection. Furthermore, the mPrickle protein which has been shown to bind to PSD-95, known as a scaffold protein, can be used to purify PSD-95. In addition, since the precipitation of endogenous mPrickle using an antibody against mPrickle results in coprecipitation of NMDA receptors, mPrickle can be used in drug delivery systems that target NMDA receptors. NMDA receptors, which are closely associated with learning and memory, are also suggested to be involved in mental disorders. Therefore, mPrickle is expected to be applicable for the diagnosis of learning- and memory-related disorders such as mental deterioration and dementia in the future. Polynucleotides of the present invention encode mPrickle and comprise the nucleotide sequence described in SEQ ID NO: 2, or a complementary sequence thereof.

Here, a “polynucleotide” refers to a polymer comprising nucleotides or nucleotide pairs of multiple deoxyribonucleic acids (DNA) or ribonucleic acids (RNA), and includes DNA, cDNA, genomic DNA, chemically synthesized DNA, and RNA. If needed, polynucleotides can also contain non-naturally-occurring nucleotides such as 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, b-D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueuosine, 5-methoxycarbonylmethyl-2-thiouridine, 5-methoxycarbonylmethyluridine, 5-methoxyuridine, 2-methylthio-N-6-isopentenyladenosine, N-((9-b-D-ribofuranosyl-2-methylthiopurin-6-yl)carbamoyl)threonine, N-((9-b-D-ribofuranosylpurin-6-yl)N-methylcarbamoyl)threonine, uridine-5-oxyacetic acid-methyl ester, uridine-5-oxyacetic acid, wybutoxosine, pseudouridine, queuosine, 2-thiocytidine, 5-methyl-2-thiouridine, 2-thiouridine, 4-thiouridine, 5-methyluridine, N-((9-b-D-ribofuranosylpurin-6-yl)carbamoyl)threonine, 2′-O-methyl-5-methyluridine, 2′-O-methyluridine, wybutosine, and 3-(3-amino-3-carboxy propyl)uridine.

Moreover, a polynucleotide of the present invention encodes mPrickle protein, and comprises a nucleotide sequence encoding the amino acid sequence described in SEQ ID NO: 1, or a complementary sequence thereof. In addition to the nucleotide sequence described in SEQ ID NO: 2, nucleotide sequences encoding such an amino acid sequences include those that differ from the sequence described in SEQ ID NO: 2 due to degeneracy of the genetic code. A polynucleotide of the present invention can be designed to express a polypeptide using genetic engineering techniques, by selecting a nucleotide sequence that has a high expression efficiency in view of the host's codon usage frequency (Grantham et al. (1981) Nucleic Acids Res. 9: 43-74).

The polynucleotides of the present invention also comprise a nucleotide sequence encoding mPrickle protein or an antigenic fragment thereof, wherein one or more amino acids in the amino sequence of SEQ ID NO: 1 are deleted, inserted, substituted, or added, or a sequence complementary to this nucleotide sequence. It is well known that a mutant polypeptide comprising an amino acid sequence, in which one or more amino acids are deleted, inserted, substituted, or added, also maintain the same biological activity as the original polypeptide (Mark et al. (1984) Proc. Natl. Acad. Sci. USA 81: 5662-6; Zoller and Smith (1982) Nucleic Acids Res. 10: 6487-500; Wang et al. (1984) Science 224: 1431-3; Dalbadie-McFarland et al. (1982) Proc. Natl. Acad. Sci. USA 79: 6409-13).

Here, an amino acid substitution refers to a mutation in which one or more amino acid residues in a sequence are changed to a different type of amino acid residue. When the amino acid sequence encoded by a polynucleotide of the present invention is altered by such a substitution, a conservative substitution is preferably carried out if the function of the protein is to be maintained. A conservative substitution means altering a sequence so that it encodes an amino acid that has properties similar to those of the amino acid before substitution. Amino acids can be classified, based on their properties, into non-polar amino acids (Ala, Ile, Leu, Met, Phe, Pro, Trp, Val), non-charged amino acids (Asn, Cys, Gln, Gly, Ser, Thr, Tyr), acidic amino acids (Asp, Glu), basic amino acids (Arg, His, Lys), neutral amino acids (Ala, Asn, Cys, Gln, Gly, Ile, Leu, Met, Phe, Pro, Ser, Thr, Trp, Tyr, Val), aliphatic amino acids (Ala, Gly), branched amino acids (Ile, Leu, Val), hydroxyamino acids (Ser, Thr), amide-type amino acids (Gln, Asn), sulfur-containing amino acids (Cys, Met), aromatic amino acids (His, Phe, Trp, Tyr), heterocyclic amino acids (His, Trp), imino acids (Pro, 4Hyp), and such. In particular, substitutions among Ala, Val, Leu, and Ile; Ser and Thr; Asp and Glu; Asn and Gln; Lys and Arg; and Phe and Tyr, are preferable in order to maintain protein properties. There are no particular limitations on the number and sites of the mutated amino acids, as long as the amino acid encoded by the polynucleotide has mPrickle antigenicity.

A polynucleotide encoding an amino acid sequence, in which one or more amino acids are deleted, inserted, substituted, or added to the sequence of SEQ ID NO: 1, can be prepared according to methods such as site-directed mutagenesis described in Molecular Cloning, A Laboratory Manual 2nd ed. (Cold Spring Harbor Press (1989), Current Protocols in Molecular Biology (John Wiley & Sons (1987-1997); especially Section8.1-8.5), Hashimoto-Goto et al. (1995) Gene 152: 271-5, Kunkel (1985) Proc. Natl. Acad. Sci. USA 82: 488-92, Kramer and Fritz (1987) Method. Enzymol. 154: 350-67, Kunkel (1988) Method. Enzymol. 85: 2763-6), and others.

Moreover, a polynucleotide of the present invention is a polynucleotide comprising a nucleotide sequence that hybridizes under stringent conditions with the nucleotide sequence of SEQ ID NO: 2, or a sequence complementary to this sequences, wherein the polynucleotide encodes mPrickle protein or an antigenic fragment thereof. Examples of such polynucleotides include isoforms, alternative isoforms, and allelic mutations, and these are also included in polynucleotides of the present invention. Such polynucleotides can be obtained from cDNA libraries or genomic libraries derived from animals such as humans, mice, rats, rabbits, hamsters, chickens, pigs, cows, goats, and sheep, by using a polynucleotide probe consisted of a nucleotide sequence comprising the nucleotide sequence of SEQ ID NO: 2 or a fragment thereof, and a known hybridization method such as colony hybridization, plaque hybridization, or Southern blotting. See “Molecular Cloning, A Laboratory Manual 2nd ed.” (Cold Spring Harbor Press (1989)) for methods of cDNA library construction. In addition, a commercially available cDNA library or genomic library may also be used.

More specifically, in constructing a cDNA library, total RNA is first prepared from cells, organs, tissues, or such that express a polynucleotide of the present invention, by known techniques such as guanidine ultracentrifugation (Chirwin et al. (1979) Biochemistry 18: 5294-5299) or AGPC (Chomczynski and Sacchi (1987) Anal. Biochem. 162: 156-159), followed by purification of mRNA using the mRNA Purification Kit (Pharmacia), or such. A kit for direct mRNA preparation, such as the QuickPrep mRNA Purification Kit (Pharmacia), may also be used. Next, cDNA is synthesized from the resulting mRNA using reverse transcriptase. cDNA synthesis kits such as the AMV Reverse Transcriptase First-strand cDNA Synthesis Kit (Seikagaku Corporation) are also commercially available. Other methods that use the 5′-RACE method to synthesize and amplify cDNA by PCR may also be used (Frohman et al. (1988) Proc. Natl. Acad. Sci. USA 85: 8998-9002; Belyavsky et al. (1989) Nucleic Acids Res. 17: 2919-32). In addition, in order to construct cDNA libraries containing a high percentage of full-length clones, known techniques such as the oligo-capping method (Maruyama and Sugano (1994) Gene 138: 171-4; Suzuki (1997) Gene 200: 149-56) can also be employed. The cDNA obtained in this manner is then incorporated into a suitable vector.

Examples of hybridization conditions in the present invention include “2×SSC, 0.1% SDS, 50° C.”, “2×SSC, 0.1% SDS, 42° C.” and “1×SSC, 0.1% SDS, 37° C.”. Examples of conditions of higher stringency include “2×SSC, 0.1% SDS, 65° C.”, “0.5×SSC, 0.1% SDS, 42° C.” and “0.2×SSC, 0.1% SDS, 65° C.”. More specifically, a method that uses the Rapid-hyb buffer (Amersham Life Science) can be carried out by performing pre-hybridization at 68° C. for 30 minutes or more, adding a probe to allow hybrid formation at 68° C. for one hour or more, washing three times in 2×SSC/0.1% SDS at room temperature for 20 minutes each, washing three times in 1×SSC/0.1% SDS at 37° C. for 20 minutes each, and finally washing twice in 1×SSC/0.1% SDS at 50° C. for 20 minutes each. This can also be carried out using, for example, the Expresshyb Hybridization Solution (CLONTECH), by performing pre-hybridization at 55° C. for 30 minutes or more, adding a labeled probe and incubating at 37° C. to 55° C. for one hour or more, washing three times in 2×SSC/0.1% SDS at room temperature for 20 minutes each, and washing once at 37° C. for 20 minutes with 1×SSC/0.1% SDS. Here, conditions of higher stringency can be achieved by increasing the temperature for pre-hybridization, hybridization, or second wash. For example, a pre-hybridization and hybridization temperature of 60° C. can be raised to 68° C. for higher stringency. In addition to factors such as temperature and salt concentration of the buffer, a person with ordinary skill in the art can also integrate other factors such as probe concentration, probe length, and reaction time, to obtain rat Prickle isoforms and allelic mutants attained in the Examples of the present invention, and corresponding genes derived from other organisms.

References such as Molecular Cloning, A Laboratory Manual 2nd ed. (Cold Spring Harbor Press (1989); Section 9.47-9.58), Current Protocols in Molecular Biology (John Wiley & Sons (1987-1997); Section 6.3-6.4), DNA Cloning 1: Core Techniques, A Practical Approach 2nd ed. (Oxford University (1995); Section2.10 for conditions, in particular), can be referred to for detailed information on hybridization procedures. Examples of hybridizing polynucleotides include polynucleotides having a nucleotide sequence that has at least 50% or more, preferably 70%, more preferably 80% and even more preferably 90% (for example, 95% or more, or 99%) identity with a nucleotide sequence comprising the nucleotide sequence of SEQ ID NO: 2. Such identities can be determined by the BLAST algorithm (Altschul (1990) Proc. Natl. Acad. Sci. USA 87: 2264-8; Karlin and Altschul (1993) Proc. Natl. Acad. Sci. USA 90: 5873-7). Examples of programs that have been developed based on this algorithm include the BLASTX program for determining the identity of amino acid sequences, and the BLASTN program for nucleotide sequences (Altschul et al. (1990) J. Mol. Biol. 215: 403-10). These programs can be used for the sequences of the present invention (see http://www.ncbi.nlm.nih.gov. for a specific example of analysis methods). Homology between the protein encoded by the mammalian Prickle gene of the present invention and D-Prickle is approximately 23% at the amino acid level.

mPrickle isoforms or allelic mutants, and other genes with mPrickle-like structure or function can be obtained from cDNA libraries and genome libraries of animals such as humans, mice, rats, rabbits, hamsters, chickens, pigs, cows, goats, and sheep, by designing primers based on the nucleic acid sequence of SEQ ID NO: 2, using gene amplification technology (PCR) (Current Protocols in Molecular Biology, John Wiley & Sons (1987) Sections 6.1-6.4).

The polynucleotide sequences of the present invention can be confirmed by using conventional sequence determination methods. For example, the dideoxynucleotide chain termination method (Sanger et al. (1977) Proc. Natl. Acad. Sci. USA 74: 5463) can be used. In addition, sequences can also be analyzed using a suitable DNA sequencer.

<Nucleotide Chains>

Moreover, a nucleotide chain complementary to a polynucleotide of the present invention comprising at least 15 nucleotides is provided by the present invention. Here, a “complementary sequence” refers to not only cases where at least 15 consecutive nucleotides of the nucleotide sequence completely pair with the template, but also includes those that have at least 70%, preferably 80%, more preferably 90% and even more preferably 95% or more (for example, 97% or 99%) of the consecutive nucleotides paired with the template. Pair formation refers to the formation of a chain, in which T (U in the case of an RNA) corresponds to A, A corresponds to T or U, G corresponds to C, and C corresponds to G in the nucleotide sequence of the template polynucleotide. Homologies can be determined by methods similar to that used in the aforementioned polynucleotide hybridization.

Such a nucleotide chain of the present invention can be used as a probe for detecting or isolating, or as a primer for amplifying, the polynucleotides of the present invention. The nucleotide chain normally consists of 15 to 100, and preferably 15 to 35 nucleotides when used as a primer, and at least 15 and preferably 30 nucleotides when used as a primer. A primer can be designed to have a restriction enzyme recognition sequence, a tag or such, added to the 5′-end side thereof, and at the 3′ end, a sequence complementary to a target sequence. A nucleotide chain of the present invention can hybridize with a polynucleotide of the present invention. Moreover, intracellular mutations of a polynucleotide of the present invention can be detected using these probes or primers. In some cases, such mutations may cause abnormalities in the activity or expression of the polypeptides of the present invention; therefore, nucleotide chains of the present inventions are thought to be useful for disease diagnosis and such.

In addition, the nucleotide chains of the present invention include antisense nucleic acids that suppress the cellular expression of a polynucleotide of the present invention by binding to an mRNA or DNA, and ribozymes that suppress via specific cleavage of mRNA.

Examples of antisense mechanisms for suppressing target gene expression include: (1) inhibition of transcription initiation via triplex formation, (2) transcription suppression through hybrid formation at sites of local open-loop structure formed by RNA polymerases, (3) transcription inhibition through hybrid formation with RNA during synthesis, (4) suppression of splicing through hybrid formation at intron-exon junctions, (5) suppression of splicing through hybrid formation at sites of spliceosome formation, (6) suppression of mRNA migration to the cytoplasm through hybrid formation with mRNA, (7) suppression of splicing through hybrid formation at a capping site or poly A addition site, (8) suppression of translation initiation through hybrid formation at the binding sites of initiation factors, (9) translation suppression through hybrid formation at ribosome binding sites, (10) suppression of peptide chain elongation through hybrid formation at mRNA coding regions or polysome binding sites, and (11) suppression of gene expression through hybrid formation at sites of nucleic acid/protein interaction (Hirashima and Inoue, “New Biochemistry Experiment Course 2, Nucleic Acids IV, Gene Replication and Expression”, Japanese Biochemical Society edit., Tokyo Kagaku Dozin Publishing, pp. 319-347 (1993)).

An antisense nucleic acid comprised in a nucleotide chain of the present invention may be a nucleic acid that inhibits gene expression by any of the mechanisms described in (1) to (11) above. Namely, it may contain an antisense sequence to not only the coding region, but also to a non-coding region sequence of a target gene whose expression is to be inhibited. A DNA that encodes an antisense nucleic acid can be used by linking to a suitable regulatory sequence that allows its expression. The antisense nucleic acid does not need to be completely complementary to the coding region or non-coding region of a target gene, as long as it can effectively inhibit the expression of the gene. Such antisense nucleic acids have a chain length of at least 15 bp or more, preferably 100 bp or more, and more preferably 500 bp or more, and are normally within 3000 bp, preferably within 2000 bp and more preferably within 1000 bp. It is preferred that such antisense nucleic acids share an identity of 90% or more, and more preferably 95% or more, with the complementary chain of a target gene transcription product. These antisense nucleic acids can be prepared according to the phosphothionate method (Stein (1988) Nucleic Acids Res. 16: 3209-3221) using the polynucleotides of the present invention.

“Ribozyme” is a generic term referring to catalysts with an RNA component, and ribozymes are broadly classified into large ribozymes and small ribozymes. Large ribozymes are enzymes that cleave the phosphate-ester bonds of a nucleic acid and leave the reaction sites with 5′-phosphoric acid and 3′-hydroxyl group at the end of a reaction. Large ribozymes are further classified into (1) group I intron RNAs, which undergo guanosine-initiated trans-esterification reactions at 5′-spliced sites, (2) group II intron RNAs, which undergo two-step self-splicing reactions with a resultant lariat structure, and (3) RNA components of ribonuclease P, which cleave precursor tRNAs at their 5′ side via hydrolysis reactions. In contrast, small ribozymes are comparatively small structural units (about 40 bp) that cleave RNAs, forming 5′-hydroxyl groups and 2′-3′ cyclic phosphoric acids. Small ribozymes include, for example, hammerhead-type ribozymes (Koizumi et al. (1988) FEBS Lett. 228: 225) and hairpin-type ribozymes (Buzayan (1986) Nature 323: 349; Kikuchi and Sasaki (1992) Nucleic Acids Res. 19: 6571; H. Kikuchi (1992) Chemistry and Biology 30: 112). Since ribozymes are easily altered and synthesized, various modification methods are known. For example, hammerhead-type ribozymes that recognize and cleave nucleotide sequence UC, UU, or UA within a target RNA can be created by designing the substrate binding portion of a ribozyme to be complementary to an RNA sequence near the target site (Koizumi et al. (1988) FEBS Lett. 228: 225; M. Koizumi and E. Ohtsuka (1990) Protein, Nucleic Acid, and Enzyme 35: 2191; Koizumi et al. (1989) Nucleic Acids Res. 17: 7059). Hairpin-type ribozymes can also be designed and produced using known methods (Kikuchi and Sasaki (1992) Nucleic Acids Res. 19: 6571; H. Kikuchi (1992) Chemistry and Biology 30: 112).

Antisense nucleic acids and ribozymes comprised in the nucleotide chains of the present invention can also be used as virus vectors derived from retroviruses, adenoviruses, adeno-associated viruses, and such, non-virus vectors that use liposomes, or naked DNAs, to control gene expression in cells using ex vivo or in vivo methods for gene therapy.

The nucleotide sequences of the nucleotide chains of the present invention can be confirmed by the same methods used for the aforementioned polynucleotides.

<Vectors>

Vectors comprising a polynucleotide of the present invention are provided by the present invention. A vector of the present invention is useful for carrying a polynucleotide of the present invention within host cells, or for expressing a polypeptide encoded by the polynucleotide. This vector includes various vectors such as plasmids, cosmids, viruses, bacteriophages, cloning vectors, and expression vectors (Molecular Cloning, A Laboratory Manual 2nd ed., Cold Spring Harbor Press (1989); Current Protocols in Molecular Biology, John Wiley & Sons (1987)). In a preferred embodiment, a polynucleotide of the present invention is expressed in a host cell, into which a vector of the present invention has been introduced, by linking to the downstream of a regulatory sequence. Here, “regulatory sequence” includes promoters, ribosome binding sites, and terminators in the case of a prokaryotic host cell, and promoters and terminators in the case of a eukaryotic host cell, and in some cases, may also contain transactivators, transcription factors, poly A signals which stabilize transcription products, splicing and polyadenylation signals, and others. Such a regulatory sequence comprises all the components required for the expression of a polynucleotide linked thereto. In addition, a vector of the present invention preferably comprises a selection marker. Moreover, a signal peptide required for transferring an intracellularly expressed polypeptide into the lumen of the endoplasmic reticulum, or the periplasm or extracellular space when the host is a Gram negative microbe, can also be incorporated into an expression vector by linking to a polypeptide of interest. Moreover, a linker may be added, and a start (ATG) or stop codon (TAA, TAG or TGA) may be inserted as necessary.

A vector of the present invention is preferably an expression vector. An “expression vector” refers to a construct capable of expressing a polypeptide encoded in an expression vector in target host cells or in vitro. The expression vectors of the present invention include cloning vectors, binary vectors, integration vectors, and such. Expression processes include transcription of the coding sequence comprised on an expression vector into translatable mRNA, translation of the mRNA into a polypeptide of the present invention, and in some cases, secretion of the expressed polypeptide into the lumen of the endoplasmic reticulum, the periplasm, or extracellular space.

pBEST (Promega) is an example of a vector capable of expressing polynucleotides in vitro. In addition, examples of promoters capable of expressing polynucleotides in prokaryotic cells such as E. coli, include PL, araB (Better et al. (1988) Science 240: 1041-3), lacZ (Ward et al. (1989) Nature 341: 544-6; Ward et al. (1992) FASEB J. 6: 2422-7), trp, tac and trc (fusion of lac and trp). In addition, terminators derived from trpA, phages, and rrnB ribosomal RNAs can also be used. Moreover, vectors to be used in E. coli preferably have an “ori” for amplifying the vector within a host, and a marker gene for selecting a transformed host. The use of a drug resistance gene is preferred, which allows the host to be distinguished by drugs such as ampicillin, tetracyclin, kanamycin, and chloramphenicol. The pelB signal sequence can be used, particularly if the polypeptide is intended for secretion into the periplasm (Lei et al. (1987) J. Bacteriol. 169: 4379). Examples include M13 vectors, pUC vectors, pBR322, pCR-Script, pGEX-5X-1 (Pharmacia), pEGFP, pBluescript (Stratagene), and pET (Invitrogen; a preferable host for this vector is BL21 expressing the T7 polymerase). In addition, subcloning or excision vectors can be exemplified by pGEM-T, pDIRECT and pT7, in particular.

An example of a bacterial host other than E. coli is the genus Bacillus, and examples of vectors include pUB110 and pc194 vectors. Specific examples include pPL608 and pKTH50 derived from Bacillus subtilis. Vectors have also been developed for host bacteria, for example, genus Pseudomonas such as Pseudomonas putida and Pseudomonas cepacia, genus Brevibacterium such as Brevibacterium lactofermentum (pAJ43 (Gene 39: 281 (1985) etc.)), genus Corynebacterium such as Corynebacterium glutamicum (pCS11 (Unexamined Published Japanese Patent Application No. Sho 57-183799); pCB11 (Mol. Gen. Genet. 196: 175 (1984), etc.)), genus Streptococcus (pHV1301 (FEMS Microbiol. Lett. 26: 239 (1985)); pGK1 (Appl. Environ. Microbiol. 50: 94 (1985)), etc.), genus Lactobacillus (pAMb1 (J. Bacteriol. 137: 614 (1979), etc.)), genus Rhodococcus such as Rhodococcus rhodochrous (J. Gen. Microbiol. 138: 1003 (1992)), and genus Streptomyces such as Streptomyces lividans and Streptomyces virginiae (see Genetic Manipulation of Streptomyces: A Laboratory Manual, Hopwood et al., Cold Spring Harbor Laboratories (1985); pIJ486 (Mol. Gen. Genet. 203: 468-478 (1986)), pKC1064 (Gene 103: 97-9 (1991)), pUWL-KS (Gene 165: 149-50 (1995))). See literatures such as “Basic Microbiology Course 8-Genetic Engineering” (Kyoritsu Publishing) for useful vectors in microbe hosts. Techniques such as the calcium chloride method (Mandel and Higa (1970) J. Mol. Biol. 53: 158-162; Hanahan (1983) J. Mol. Biol. 166: 557-580) and electroporation can be employed to introduce a vector into a host.

Further, regulatory elements for expression in eukaryotic cell hosts are exemplified by the AOX1 and GAL1 promoters for yeast hosts. Examples of expression vectors derived from yeasts include the Pichia Expression Kit (Invitrogen), pNV11 and SP-Q01. Vectors that can be used in yeasts are described in detail in, for example, Adv. Biochem. Eng. 43: 75-102 (1990) and Yeast 8: 423-88 (1992). More specifically, vectors such as YRp, YEp, YCp, and YIp can be used in genus Saccharomyces such as Saccharomyces cerevisiae. Integration vectors (such as EP537456), which allow a large number of gene copies to be inserted, and can stably maintain the inserted genes, are particularly useful. Other examples of vectors include 2 μm vectors derived from S. cerevisiae, pKD1 vectors (J. Bacteriol. 145: 382-90 (1981), pGK11-derived vectors, and Kluyveromyces autonomous replication gene KARS vectors for genus Kluyveromyces such as Kluyveromyces lactis; vectors described in Mol. Cell. Biol. 6: 80 (1986) and pAUR224 (Takara Shuzo) for genus Schizosaccharomyces; pSB3-derived vectors (Nucleic Acids Res. 13: 4267 (1985)) for genus Zygosaccharomyces; vectors described in literatures such as Yeast 7: 431-43 (1991), Mol. Cell. Biol. 5: 3376 (1985) and Nucleic Acids Res. 15: 3859 (1987) for genus Pichia such as Pichia angusta and Pichia pastoris; vectors described in Unexamined Published Japanese Patent Application No. Hei 8-173170 or vectors using ARS derived from Candida maltosa (Agri. Biol. Chem. 51: 1587 (1987)) for genus Candida such as C. maltosa, C. albicans, C. tropicalis or C. utilis; vectors described in Trends in Biotechnology 7: 283-7 (1989) for genus Aspergillus such as Aspergillus niger and A. oryzae; and vectors using promoters derived from the extracellular cellulase gene (Bio/technology 7: 596-603 (1989)) in genus Trichoderma.

For hosts of mammalian cells or other animal cells, the adenovirus late promoter (Kaufman et al. (1989) Mol. Cell. Biol. 9: 946), CAG promoter (Niwa et al. (1991) Gene 108: 193-200), CMV immediate-early promoter (Seed and Aruffo (1987) Proc. Natl. Acad. Sci. USA 84: 3365-9), EF1α promoter (Mizushima et al. (1990) Nucleic Acids Res. 18: 5322; Kim et al. (1990) Gene 91: 217-23), HSV TK promoter, SRα promoter (Takebe et al. (1988) Mol. Cell. Biol. 8: 466), SV40 promoter (Mulligan et al. (1979) Nature 277: 108), SV40 early promoter (Genetic Engineering Vol. 3, Williamson ed., Academic Press (1982) pp. 83-141), SV40 late promoter (Gheysen and Fiers (1982) J. Mol. Appl. Genet. 1: 385-94), RSV (Rous sarcoma virus)-LTR promoter (Cullen (1987) Methods Enzymol. 152: 684-704), MMLV-LTR promoter, CMV enhancer, SV40 enhancer and globin intron, and such can be used. Moreover, the vector preferably comprises a drug resistance gene to allow cells to be distinguished by drugs such as neomycin or G418. To increase the number of gene copies within cells, the number of copies can be amplified by using methotrexate (MTX) in, for example, a CHO host which is defective in the nucleic acid synthesis pathway, and employing a vector such as pCHOI, which has a DHFR gene to compensate for the defect. On the other hand, in order to transiently express a gene, COS cells having an SV40 T antigen gene on their chromosomes can be used as the host, and a vector having an SV40 replication origin, such as pcD, or a vector having a replication origin of adenovirus, bovine papilloma virus (BPV), polyoma virus, and such can be used. Moreover, a gene encoding aminoglycoside transferase (APH), thymidine kinase (TK), xanthine-guanine phosphoribosyl transferase (Ecogpt), dihydrofolic acid reductase (dhfr), or such may be included as a selection marker for amplifying the gene copy number. Known examples of suitable vectors are the Okayama-Berg expression vector pcDV1 (Pharmacia), pCDM8 (Nature 329: 840-2 (1987)), pRc/CMV, pcDNA1, pcDNA3 (Invitrogen), pSPORT1 (GIBCO BRL), pSV2dhfr (Mol. Cell. Biol. 1: 854-64 (1981)), pEF-BOS (Nucleic Acids Res. 18: 5322 (1990)), pCEP4 (Invitrogen), pMAM, pDR2, pBK-RSV, pBK-CMV, pOPRSV, pOP13, and pME18S (Mol. Cell. Biol. 8: 466-72 (1988)).

In particular, examples of vectors used to express a polynucleotide of the present invention in animals in vivo include adenovirus vectors such as pAdexlcw and retrovirus vectors such as pZlPneo. A vector can be introduced into a host using methods such as adenovirus methods, electroporation (Cytotechnology 3: 133 (1990)), cationic liposome methods (Cationic Liposome DOTAP (Boehringer Mannheim), etc.), introduction using positively charged polymers, electrostatic type liposome methods, internal type liposome methods, particle gun methods, liposome methods, lipofection (Proc. Natl. Acad. Sci. USA 84: 7413 (1987)), calcium phosphate methods (Unexamined Published Japanese Patent Application No. Hei 2-227075), receptor-mediated gene introduction methods, retrovirus methods, DEAE dextran methods, virus-liposome methods (Experimental Medicine Supplement, “Basic Technology of Gene Therapy”, Yodosha (1997); Experimental Medicine Supplement, “Experimental Method of Gene Introduction and Expression Analysis”, Yodosha (1997); J. Clin. Invest. 93: 1458-64 (1994); Am. J. Physiol. 271: R1212-20 (1996); Molecular Medicine 30: 1440-8 (1993); Experimental Medicine 12: 1822-6 (1994); Protein, Nucleic acid, and Enzyme 42: 1806-13 (1997); Circulation 92 (Suppl. II): 479-82 (1995)), and naked-DNA direct introduction methods. Vectors generated using virus vectors derived from viruses other than adenoviruses and retroviruses, such as adeno-associated virus, Sindbis virus, Sendai virus, Togavirus, Paramyxovirus, poxvirus, poliovirus, herpes virus, lentivirus and vaccinia virus, can also be used. Administration into the living body may be carried out using ex vivo or in vivo methods.

In addition, insect expression systems are also known as systems for expressing heterogeneous polypeptides. For example, exogenous genes can be expressed in Spodoptera frugiperda cells or Trichoplusia larvae cells, using the Autographa california nucleopolyhedrosis virus (AcNPV) as a vector. Here, an exogenous gene of interest is cloned into the non-essential region of a virus. For example, it may be linked to a region under the control of a polyhedrin promoter. In this case, the polyhedrin gene is deactivated, a recombinant virus lacking the coat protein is produced, and a polypeptide of interest is expressed in cells of Spodoptera frugiperda, Trichoplusia larvae, or such, that have been infected with the virus (Smith (1983) J. Virol. 46: 584; Engelhard (1994) Proc. Natl. Acad. Sci. USA 91: 3224-7). Other known examples of insect cell-derived expression vectors include the Bac-to-BAC Baculovirus Expression System (Bigco BRL) and pBacPAK8.

When plant cells are used as a host, for example, vectors that use the 35S promoter of cauliflower mosaic virus can be used. Known methods of introducing a vector into plant cells include the PEG, electroporation, Agrobacterium methods, and particle gun methods.

Insertion of a DNA into a vector can be carried out in a ligase reaction using restriction enzyme sites (Current Protocols in Molecular Biology, John Wiley & Sons (1987) Section 11.4-11.11; Molecular Cloning, A Laboratory Manual 2nd ed., Cold Spring Harbor Press (1989) Section 5.61-5.63).

<Hosts>

The present invention provides hosts that comprise a polynucleotide or vector of the present invention. An in vitro or in vivo production system may be employed for the production of a polypeptide of the present invention. Hosts of the present invention include archaebacterial, bacterial, fungal, plant, insect, fish, amphibian, reptilian, avian, and mammalian prokaryotic and eukaryotic cells. A host of the present invention comprises in its cells a polynucleotide that encodes a polypeptide of the present invention. As long as the polynucleotide does not exist at a naturally occurring position in the genome of a host cell, the polynucleotide may be regulated by its own promoter, incorporated into the host genome, or maintained as an extrachromosomal structure.

Examples of bacterial hosts include Gram positive and Gram negative bacteria belonging to the genus Escherichia, Streptococcus, Staphylococcus, Serratia or Bacillus, such as E. coli (JM109, DH5a, HB11 and XL1Blue), Serratia marcescens, and Bacillus subtilis.

Examples of a eukaryotic host include fungal cells such as yeasts, higher plants (Nicotiana tabacum derived cells), insects (Drosophila S2, Spodoptera Sf9, Sf21, Tn5), fish, amphibians (Xenopus oocytes (Valle et al. (1981) Nature 291: 358-40), reptiles, birds, and mammals (CHO (J. Exp. Med. 108: 945 (1995). Among them, DHFR gene-deficient dhfr-CHO (Proc. Natl. Acad. Sci. USA 77: 4216-20 (1980) and CHO K-1 (Proc. Natl. Acad. Sci. USA 60: 1275 (1968)), COS, Hela, C127, 3T3, BHK, HEK293 and Bowes melanoma cells), myeloma, Vero, Namalwa, Namalwa KJM-1 and HBT5637 (Unexamined Published Japanese Patent Application No. Sho 63-299), and plants (potato, tobacco, corn, rice, rape, soybean, tomato, wheat, barley, rye, alfalfa, and hemp), are included. In addition to yeasts such as Saccharomyces cerevisiae belonging to the genus Saccharomyces, and the members of the genus Pichia, expression systems that use fungi as a host, such as the cells of Aspergillus niger belonging to the mold Aspergillus, are also known.

Introduction of a vector into host cells can be carried out using methods such as the electroporation (Chu et al. (1987) Nucleic Acids Res. 15: 1311-26), cationic liposome methods, electric pulse terebration (Current Protocols in Molecular Biology, John Wiley & Sons (1987) Sections 9.1 to 9.9), direct injection using a microscopic glass tube, microinjection, lipofection (Derijard (1994) Cell 7: 1025-37; Lamb (1993) Nature Genetics 5: 22-30; Rabindran et al. (1993) Science 259: 230-4), lipofectamine method (GIBCO-BRL), calcium phosphate method (Chen and Okayama (1987) Mol. Cell. Biol. 7: 2745-52), DEAE dextran method (Lopata et al. (1984) Nucleic Acids Res. 12: 5707-17; Sussman and Milman (1985) Mol. Cell. Biol. 4: 1642-3) and FuGene6 reagent (Boehringer-Mannheim).

<Polypeptides and Polypeptide Fragments>

A “polypeptide fragment” of the present invention refers to a peptide polymer that is a fragment of a polypeptide encoded by a polynucleotide of the present invention. A polypeptide fragment of the present invention is identical to a portion of the polypeptide comprising the amino acid sequence described in SEQ ID NO: 1, and comprises at least eight amino acid residues or more (for example, 8, 10, 12 or 15 amino acid residues or more). A particularly preferable fragment can be exemplified by a polypeptide fragment lacking an amino terminus, carboxyl terminus, and transmembrane domain. The polypeptide fragments of the present invention include fragments containing an α-helix and α-helix forming region, α-amphipathic region, β-sheet and β-sheet forming region, β-amphipathic region, substrate binding region, high antigen index region, coil and coil forming region, hydrophilic region, hydrophobic region, turn and turn forming region, and surface forming region. A polypeptide fragment of the present invention may be any fragment, provided that it has the antigenicity of a polypeptide of the present invention. The antigen-determining site of a polypeptide can be predicted using methods for analyzing protein hydrophobicity and hydrophilicity of an amino acid sequence (Kyte-Doolittle (1982) J. Mol. Biol. 157: 105-22), or methods of secondary structure analysis (Chou-Fasman (1978) Ann. Rev. Biochem. 47: 251-76), and can be confirmed using a computer program (Anal. Biochem. 151: 540-6 (1985), or the PEPSCAN method in which a short peptide is synthesized followed by confirmation of its antigenicity (Published Japanese Translation of International Publication No. Sho 60-500684).

The polypeptide fragments of the present invention may comprise naturally occurring or modified amino acid residues. Examples of amino acid residue modifications include acylation, acetylation, amidation, arginylation, GPI anchor formation, crosslinking, γ-carboxylation, cyclization, covalent crosslink formation, glycosylation, oxidation, covalent bonding of a lipid or fat derivative, cystine formation, disulfide bond formation, selenoylation, demethylation, protein fragmentation treatment, covalent bonding of a nucleotide or nucleotide derivative, hydroxylation, pyroglutamate formation, covalent bonding of a flavin, prenylation, covalent bonding with a heme portion, covalent bonding of phosphatidyl inositol, formylation, myristoylation, methylation, ubiquitination, iodination, racemization, ADP-ribosylation, sulfation and phosphorylation. Moreover, the polypeptides of the present invention include precursors containing a signal peptide portion, mature proteins lacking a signal peptide portion, and fusion proteins modified with other peptide sequences. Peptide sequences to be added to a polypeptide of the present invention can be selected from sequences that facilitate protein purification using, for example, pcDNA3.1/Myc-His vector (Invitrogen), or those that confer stability in recombinant protein production. Examples of such sequences are influenza agglutinin (HA), glutathione S transferase (GST), substance P, multiple histidine tag (such as 6× His and 10× His), protein C fragment, maltose-binding protein (MBP), immunoglobulin constant region, a-tubulin fragment, b-galactosidase, B-tag, c-myc fragment, E-tag (epitope on a monoclonal phage), FLAG (Hopp et al. (1988) Bio/Technol. 6: 1204-10), lck tag, p18 HIV fragment, HSV-tag (human simple Herpes virus glycoprotein), SV40T antigen fragment, T7-tag (T7 gene 10 protein), and VSV-GP fragment (vesicular stomatitis virus glycoprotein).

The polypeptide fragments of the present invention can be produced using known genetic recombination techniques or chemical synthesis methods. When the polypeptides of this invention or fragments thereof are produced using genetic recombination techniques, the resultant proteins may or may not be glycosylated. Further, they may have different molecular weights, isoelectric points, and so on, depending on the host selected. In general, when polypeptides are expressed in a prokaryotic host such as Escherichia coli, the resultant polypeptides are produced in a form having a methionine residue added to the N terminus of the original polypeptides. The polypeptides of the present invention also include polypeptides that have different structures as a result of different hosts.

<Polypeptide Production>

For in vitro production of polypeptides or polypeptide fragments, polypeptides can be produced in an in vitro cell-free system using methods such as in vitro translation (Dasso and Jackson (1989) Nucleic Acids Res. 17: 3129-44). In contrast, when producing polypeptides using cells, a suitable cell host is first selected from those mentioned above, and then the cells are transformed with a DNA of interest. Subsequently, the transformed cells can be cultured to obtain a polypeptide of interest. Culturing is carried out using known methods that are appropriate for the cell host selected. For example, when animal cells are selected, culturing can be carried out at a pH of about 6 to 8 and a temperature of 30° C. to 40° C. for about 15 to 200 hours, using a medium such as DMEM (Virology 8: 396 (1959)), MEM (Science 122: 501 (1952)), RPM11640 (J. Am. Med. Assoc. 199: 519 (1967)), 199 (Proc. Soc. Biol. Med. 73: 1 (1950)) or IMDM, and adding serum such as fetal calf serum (FCS), as necessary. In addition, the medium may be replaced, aerated, or stirred, during the course of culturing, as necessary.

On the other hand, in order to establish an in vivo polypeptide production system, a DNA of interest is introduced into an animal or plant, and the polypeptide is produced in vivo. Examples of known animal systems (Lubon (1998) Biotechnol. Annu. Rev. 4: 1-54) include mammals such as goats, pigs, sheep, mice, and cows, and insects such as silkworms (Susumu (1985) Nature 315: 592-4). In addition, transgenic animals can also be used in mammalian systems.

For example, when secreting a polypeptide of interest in goat milk, a DNA that encodes the polypeptide is linked to a DNA that encodes a protein such as β-casein, and a fusion protein of the polypeptide of interest is specifically expressed in milk. Next, the DNA that encodes the fusion protein is introduced into a goat embryo. The embryo harboring this DNA is then transferred back into the uterus of a female goat. The transgenic goats or their offspring born from this female goat secretes the polypeptide of interest in their milk. Hormones may also be administered to increase the amount of milk, as necessary (Ebert et al. (1994) Bio/Technology 12: 699-702).

Transgenic plant polypeptide production systems using plants such as tobacco are known. First, a DNA that encodes a polypeptide of interest is incorporated into a plant expression vector such as pMON530, and this vector is then introduced into a bacterium such as Agrobacterium tumefaciens. A bacterium harboring this DNA is then used to infect plants such as Nicotina tabacum, and the polypeptide of interest can be isolated from the leaves of the resulting transgenic plant upon regeneration of the plant body (Julian et al. (1994) Eur. J. Immunol. 24: 131-8). Examples of other established methods include methods in which a DNA is introduced into a protoplast using PEG followed by regeneration of the plant body (Gene Transfer to Plants, Potrykus and Spangenberg ed. (1995) 66-74; suitable for Indian rice varieties), methods in which a DNA is introduced into a protoplast by electric pulse followed by regeneration of the plant body (Toki et al. (1992) Plant Physiol. 100: 1503-7; suitable for Japanese rice varieties), methods in which a DNA is directly introduced into plant cells using the particle gun method followed by regeneration of the plant body (Christou et al. (1991) Bio/Technology 9: 957-62), and methods in which a DNA is introduced into cells via Agrobacterium followed by regeneration of the plant body (Hiei et al. (1994) Plant J. 6: 271-82). See Toki et al. (1995) Plant Physiol. 100: 1503-7 for methods of plant regeneration.

Once a transgenic plant is obtained, a plant host that produces a polypeptide of the present invention can be propagated in the same manner, using the seeds, fruits, tubers, root tubers, stocks, cuttings, calluses, or protoplasts of the plant.

Normally, a polypeptide of the present invention produced by gene recombination techniques can be recovered from the medium if the polypeptide is secreted outside of a cell, or from the body fluid of a transgenic organism. When a polypeptide is produced inside of a cell, the cells are dissolved and the polypeptide is recovered from the dissolved product. The polypeptide of interest is then purified by suitably combining known methods of protein purification such as salting out, distillation, various types of chromatography, gel electrophoresis, gel filtration, ultrafiltration, recrystallization, acid extraction, dialysis, immunoprecipitation, solvent precipitation, solvent extraction, and ammonium sulfate or ethanol precipitation. Examples of chromatographies include ion exchange chromatography, such as anion or cation exchange chromatography, affinity chromatography, reversed-phase chromatography, adsorption chromatography, gel filtration chromatography, hydrophobic chromatography, hydroxyapatite chromatography, phosphocellulose chromatography, and lectin chromatography (Strategies for Protein Purification and Characterization: A Laboratory Course Manual, Marshak et al. ed., Cold Spring Harbor Laboratory Press (1996)). Chromatography can be carried out using a liquid phase chromatography such as HPLC or FPLC.

In addition, naturally-occurring polypeptides can also be purified and obtained. For example, polypeptides can be purified by affinity chromatography using antibodies against the polypeptides of the present invention to be described below (Current Protocols in Molecular Biology, John Wiley & Sons (1987) Section 16.1-16.19). In addition, purification can also be carried out using a glutathione column for GST-fusion proteins, or a nickel column for histidine-tagged fusion proteins. When producing a polypeptide of the present invention in the form of a fusion protein, unwanted portions can be cleaved using thrombin or factor Xa and such, following purification, as necessary. Moreover, the resulting polypeptide can also be modified using enzymes such as chymotrypsin, glucosidase, trypsin, protein kinase, and lysyl endopeptidase, as necessary.

In addition to the aforementioned synthesis and genetic engineering techniques, a polypeptide fragment of the present invention can also be produced by cleaving a polypeptide of the present invention, using suitable enzymes such as peptidase.

<Antibodies>

The present invention also provides antibodies against the polypeptides or polypeptide fragments of the present invention. Antibodies of the present invention also include polyclonal antibodies, monoclonal antibodies, chimeric antibodies, single-chain antibodies (scFV) (Huston et al. (1988) Proc. Natl. Acad. Sci. USA 85: 5879-83; The Pharmacology of Monoclonal Antibody, vol. 113, Rosenburg and Moore ed., Springer Verlag (1994) 269-315), humanized antibodies, multispecific antibodies (LeDoussal et al. (1992) Int. J. Cancer Suppl. 7: 58-62; Paulus (1985) Behring Inst. Mitt. 78: 118-32; Millstein and Cuello (1983) Nature 305: 537-9; Zimmermann (1986) Rev. Physiol. Biochem. Pharmacol. 105: 176-260; Van Dijk et al. (1989) Int. J. Cancer 43: 944-9), and antibody fragments such as Fab, Fab′, F(ab′)2, Fc, and Fv. Moreover, an antibody of the present invention may also be modified by PEG and such, as necessary. An antibody of the present invention may also be produced in the form of a fusion protein with β-galactosidase, maltose-binding protein, GST, green fluorescent protein (GFP), or such, to allow detection without the use of a secondary antibody. In addition, an antibody may be modified by labeling with biotin or such to allow recovery using avidin, streptoavidin, or such.

An antibody of the present invention can be produced using a polypeptide the present invention, a fragment thereof, or cells in which a polypeptide or polypeptide fragment of the present invention is expressed, as a sensitized antigen. In addition, a short polypeptide of the present invention, or a fragment thereof, may also be used as an immunogen by coupling to a carrier such as bovine serum albumin, Keyhole-limpet hemocyanin, and ovalbumin. In addition, a polypeptide of the present invention, or a fragment thereof, may be used in combination with a known adjuvant such as aluminum adjuvant, Freund's complete (or incomplete) adjuvant, or pertussis adjuvant, to enhance the immune response to an antigen.

Polyclonal antibodies can be obtained from, for example, the serum of an immunized animal after immunizing a mammal with a polypeptide of the present invention, or a fragment thereof, coupled to a desired adjuvant. Although there are no particular limitations on the mammals used, typical examples include rodents, lagomorphs, and primates. Specific examples include rodents such as mice, rats and hamsters, lagomorphs such as rabbits, and primates such as monkeys, including cynomolgus monkeys, rhesus monkeys, baboons and chimpanzees. Animal immunization is carried out by suitably diluting and suspending a sensitized antigen in phosphate-buffered saline (PBS) or physiological saline, mixing with an adjuvant as necessary until emulsified, and injecting into an animal intraperitoneally or subcutaneously. The sensitized antigen mixed with Freund's incomplete adjuvant is preferably administered several times, every 4 to 21 days. Antibody production can be confirmed by measuring the level of an antibody of interest in the serum using conventional methods. Finally, the serum itself may be used as a polyclonal antibody, or it may be further purified. See, for example, “Current Protocols in Molecular Biology” (John Wiley & Sons (1987) Sections 11.12-11.13), for specific methods.

A monoclonal antibody can be produced by removing the spleen from an animal immunized in the manner described above, separating immunocytes from the spleen, and fusing with a suitable myeloma cell using polyethylene glycol (PEG) or such to establish hybridomas. Cell fusion can be carried out according to the Milstein method (Galfre and Milstein (1981) Methods Enzymol. 73: 3-46). Here, suitable myeloma cells are exemplified particularly by cells that allow chemical selection of fused cells. When using such myeloma cells, fused hybridomas are selected by culturing in a culture medium (HAT culture medium) that contains hypoxanthine, aminopterin and thymidine, which destroy cells other than the fused cells. Next, a clone that produces an antibody against a polypeptide of the present invention, or a fragment thereof, is selected from the established hybridomas. Subsequently, the selected clone is introduced into the abdominal cavity of a mouse or such, and ascites is collected to obtain a monoclonal antibody. See, in addition, “Current Protocols in Molecular Biology” (John Wiley & Sons (1987) Section 11.4-11.11), for information on specific methods.

Hybridomas can also be obtained by first sensitizing human lymphocytes that have been infected by EB virus with an immunogen in vitro, and fusing the sensitized lymphocytes with human myeloma cells (such as U266) to obtain hybridomas that produce human antibodies (Unexamined Published Japanese Patent Application No. Sho 63-17688). In addition, human antibodies can also be obtained by using antibody-producing cells generated by sensitizing a transgenic animal with a human antibody gene repertoire (WO92/03918; WO93-02227; WO94/02602; WO94/25585; WO96/33735; WO96/34096; Mendez et al. (1997) Nat. Genet. 15: 146-156, etc.). Methods that do not use hybridomas can be exemplified by a method in which a cancer gene is introduced to immortalize immunocytes such as antibody producing lymphocytes.

In addition, antibodies can also be produced by genetic recombination techniques (see Borrebaeck and Larrick (1990) Therapeutic Monoclonal Antibodies, MacMillan Publishers Ltd., UK). First, a gene that encodes an antibody is cloned from hybridomas or antibody-producing cells (such as sensitized lymphocytes). The resulting gene is then inserted into a suitable vector, the vector is introduced into a host, and the host is then cultured to produce the antibody. This type of recombinant antibody is also included in the antibodies of the present invention. Typical examples of recombinant antibodies include chimeric antibodies comprising a non-human antibody-derived variable region and a human antibody-derived constant region, and humanized antibodies comprising a non-human-derived antibody complementarity determining region (CDR), human antibody-derived framework region (FR), and human antibody constant region (Jones et al. (1986) Nature 321: 522-5; Reichmann et al. (1988) Nature 332: 323-9; Presta (1992) Curr. Op. Struct. Biol. 2: 593-6; Methods Enzymol. 203: 99-121 (1991)).

Antibody fragments of the present invention can be produced by treating the aforementioned polyclonal or monoclonal antibodies with enzymes such as papain or pepsin. Alternatively, an antibody fragment can be produced by genetic engineering techniques using a gene that encodes an antibody fragment (see Co et al., (1994) J. Immunol. 152: 2968-76; Better and Horwitz (1989) Methods Enzymol. 178: 476-96; Pluckthun and Skerra (1989) Methods Enzymol. 178: 497-515; Lamoyi (1986) Methods Enzymol. 121: 652-63; Rousseaux et al. (1986) 121: 663-9; Bird and Walker (1991) Trends Biotechnol. 9: 132-7).

The multispecific antibodies of the present invention include bispecific antibodies (BsAb), diabodies (Db), and such. Multispecific antibodies can be produced by methods such as (1) chemically coupling antibodies having different specificities with different types of bifunctional linkers (Paulus (1985) Behring Inst. Mill. 78: 118-32), (2) fusing hybridomas that secrete different monoclonal antibodies (Millstein and Cuello (1983) Nature 305: 537-9), or (3) transfecting eukaryotic cell expression systems, such as mouse myeloma cells, with a light chain gene and a heavy chain gene of different polyclonal antibodies (four types of DNA), followed by the isolation of a bispecific monovalent portion (Zimmermann (1986) Rev. Physio. Biochem. Pharmacol. 105: 176-260; Van Dijk et al. (1989) Int. J. Cancer 43: 944-9). On the other hand, diabodies are dimer antibody fragments comprising two bivalent polypeptide chains that can be constructed by gene fusion. These can be produced using known methods (see Holliger et al. (1993) Proc. Natl. Acad. Sci. USA 90: 6444-8; EP404097; WO93/11161).

Recovery and purification of antibodies and antibody fragments can be carried out using Protein A and Protein G, or according to the protein purification techniques described in detail under the above section “Polypeptide Production” (Antibodies: A Laboratory Manual, Ed Harlow and David Lane, Cold Spring Harbor Laboratory (1988)). For example, when using Protein A to purify an antibody of the present invention, known Protein A columns such as Hyper D, POROS or Sepharose F.F. (Pharmacia) can be used. The concentration of the resulting antibody can be determined by measuring the absorbance or by enzyme linked immunoadsorbent assay (ELISA).

Antigen binding activity of an antibody can be determined by absorbance measurement, or by using fluorescent antibody methods, enzyme immunoassay (EIA) methods, radioimmunoassay (RIA) methods, or ELISA. When ELISA is used, an antibody of the present invention is first immobilized onto a support such as a plate. A polypeptide of the present invention is added, and then a sample containing the antibody of interest is added. Here, samples containing an antibody of interest include, for example, culture supernatants of antibody-producing cells, purified antibodies, and such. Next, a secondary antibody that recognizes an antibody of the present invention is added, followed by the incubation of the plate. Subsequently, the plate is washed and the label attached to the secondary antibody is detected. Namely, if a secondary antibody is labeled with alkaline phosphatase, the antigen binding activity can be determined by adding an enzyme substrate such as p-nitrophenyl phosphate, and measuring the absorbance. In addition, a commercially available system such as BIAcore (Pharmacia) can also be used to evaluate antibody activities.

The antibodies of the present invention can be used to purify the polypeptides of this invention or fragments thereof. The antibodies can also be used in drug delivery systems that target NMDA receptors. Furthermore, since the localization of mPrickle protein in synapses has been confirmed by the present invention, the antibodies of this invention can also be used to detect mPrickle protein, which serves as a synaptic marker. Accordingly, the antibodies of this invention can be used as a reagent for detecting synapses as needed.

<Analysis of mPrickle Gene Expression Region>

The present invention provides an expression regulatory region of mPrickle gene. An expression regulatory region of the present invention can be cloned from genomic DNA by known methods using a polynucleotide of the present invention. For example, a method for establishing the transcription origin, such as the SI mapping method, is known and can be used in the present invention (Cell Engineering, Supplement 8, New Cell Engineering Experiment Protocol, Cancer Research Division, The Institute of Medical Science, The University of Tokyo ed., Shujunsha Publishing (1993) pp. 362-374). In general, the expression regulatory region of a gene can be cloned by screening a genomic DNA library, using a probe DNA comprising a 15-100 bp segment, and preferably a 30-50 bp segment, of the gene's 5′ terminus (in the present invention, all or a portion of nucleotides of SEQ ID NO: 2). A clone obtained in this manner contains a 5′ non-coding region of 10 kbp or more, and is shortened or fragmented by exonuclease treatment, or such. Finally, the shortened sequence portion comprising a potential expression regulatory region is evaluated for its expression, strength, regulation, and such, using a reporter gene, thereby making it possible to determine the minimum unit required for maintaining the activity of the mPrickle gene expression regulatory region of the present invention.

Gene expression regulatory regions can be predicted using a program such as Neural Network (http://www.fruitfly.org./seq_tools/promoter.html; Reese et al., Biocomputing: Proceedings of the 1996 Pacific Symposium, Hunter and Klein ed., World Scientific Publishing Co., Singapore, (1996)). Moreover, a program for predicting the minimum unit required for the activity of an expression regulatory region is also known, (http://biosci.cbs.umn.edu./software/proscan/promoterscan.htm; Prestridge (1995) J. Mol. Biol. 249: 923-932), and can be used in the present invention.

The expression regulatory region of the mPrickle gene isolated in this manner can be used to produce a protein of interest specific for postsynaptic densities in vivo.

<Ligand Identification>

The present invention provides ligands against the mPrickle protein. Because the mPrickle protein possesses LIM and PET domains, it is thought to have a nucleic acid-binding function in nature. In addition, the mPrickle protein is also expected to bind to PSD-95, which is known to be a scaffold protein, and to associate with NMDA receptors. Therefore, potential ligands that demonstrate an agonistic or antagonistic function towards the mPrickle protein may be used to regulate morphological changes in neurons that are involved in learning and/or memory, or neuronal functions such as neuronal plasticity. In identifying ligands for the mPrickle protein, a candidate compound and the mPrickle protein are first contacted and assayed for the presence of binding. Here, the mPrickle protein can be used when immobilized on a support, or expressed to be embedded in the cell membrane. There are no particular limitations on the candidate compounds, examples of which include expression products of gene libraries, natural substances derived from marine organisms, extracts of various types of cells, known compounds and peptides, natural substances derived from plants, body tissue extracts, microbial culture supernatants, and peptide groups randomly produced by the phage display method (J. Mol. Biol. 222: 301-10 (1991)). In particular, when hippocampus extract obtained by surfactant treatment is used as a candidate compound, it becomes possible to identify substances that interact with Prickle proteins in vivo. Furthermore, the candidate compound may be labeled to facilitate the detection of binding.

### BRIEF DESCRIPTION OF THE DRAWINGS

FIG. 1 is a photograph showing the results of Western blotting performed on the fraction before solubilization, and the supernatant and precipitate after solubilization, using NMDAR, PSD-95, homer lb/c, and MAPK antibodies.

FIG. 2 shows the comparison between the R-Prickle of the present invention and D-Prickle at the amino acid sequence level.

FIG. 3 is a continuation of FIG. 2.

FIG. 4 is a schematic diagram comparing domain structures in the Prickle family. Shaded areas indicate the PET domain and black areas indicate the LIM domain. Dotted areas are the regions identified by mass spectrometry. Hs: Humans, XP: Xenopus, D: Drosophila, Ci: Ciona intestinalis.

FIG. 5 is a photograph showing the histological distribution of R-Prickle in rat tissues.

FIG. 6 is a photograph showing the intracellular distribution of R-Prickle in rat brain. H: homogenate, SPM: synaptic plasma membrane, PSD-S1: 0.5% TritonX-100 soluble fraction of SPM, PSD-P1: 0.5% TritonX-100 insoluble fraction of SPM, PSD-S2: 1% TritonX-100 soluble fraction of SPM, PSD-P2: 1% TritonX-100 insoluble fraction of SPM.

FIG. 7 is a photograph showing the results of examining R-Prickle solubility in various surfactants. P: pellet, S: supernatant, Triton: TritonX-100, DOC: sodium deoxycholate.

FIG. 8 is a photograph showing the R-Prickle expression in rat brain at each developmental stage.

FIG. 9 is a photograph showing the R-Prickle localization in primary cultures of hippocampal neurons derived from rat embryos.

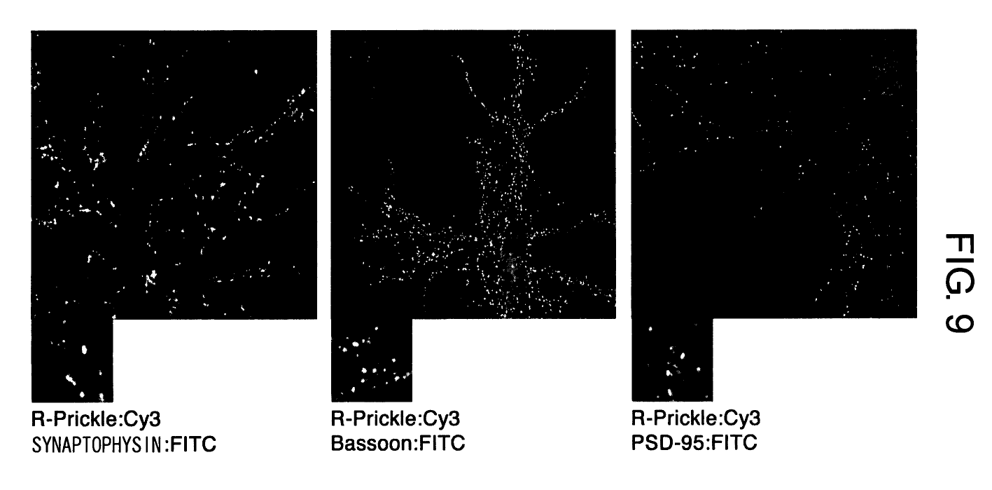

### BEST MODE FOR CARRYING OUT THE INVENTION

Herein below, the present invention will be investigated in detail using examples, however, it is not to be construed as being limited thereto.

### Example 1

### Methods for Preparing PSD Fractions

The PSD fraction was prepared from a female rat brain (Japan SLC Inc.) according to the method described by Satoh et al. (Satoh et al. (2002) “Identification of activity-regulated proteins in the postsynaptic density fraction.” Genes Cells. 7: 187-97).

### Example 2

### Concentration and Identification of Molecules with Poor Solubility

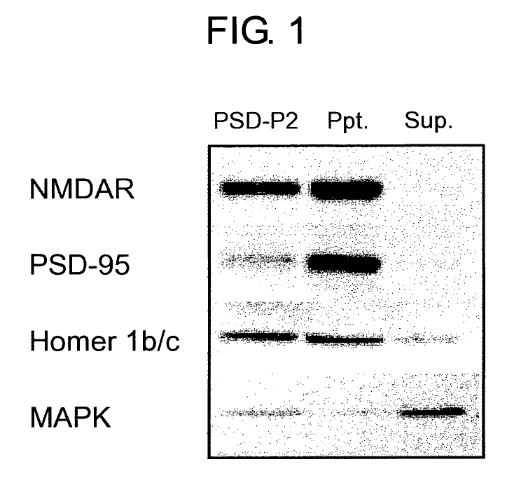

Scaffolding molecules and membrane proteins at PSDs strongly bind to cytoskeletal molecules and such to form large complexes, and therefore have poor solubilities. Thus, 2% TritonX-114 (Nacalai Tesque 355-22CP) was added to the PSD fraction obtained in Example 1, and was centrifuged at 196,000×g for 30 minutes at 4° C. to separate the soluble and insoluble fractions. Western blotting was then performed on the PSD fraction before solubilization, as well as on the soluble and insoluble fractions after solubilization, by using antibodies against the NMDA receptor, PSD-95, homer lb/c (a dendritic protein that comprises a PDZ-like binding domain and that specifically binds to the C-terminus of metabotropic glutamate receptor), and MAP (mitogen-activated protein) kinase (Santa cruz (C-29) SC-1467; Transduction laboratories, P43520; Santa cruz (C-16) SC-8923; Transduction laboratories, E17120). These results showed that the poorly soluble PSD constituent molecules have been concentrated in the TritonX-114 insoluble fraction (FIG. 1).

### Example 3

### Isolation and Identification of the TritonX-114 Insoluble Fraction

The TritonX-114 insoluble fraction of Example 2 was further solubilized in a solution containing 7M urea and 2M thiourea, separated using the MonoQ column (Amersham Pharmacia Biotech) and 1D-PAGE, and then subjected to silver staining and Western blotting. In Western blotting, the PSD-95 antibody (Transduction laboratories, P43520), GluR2/3 antibody (Chemicon MAB397), Horner lb/c antibody (Santa cruz (C-16) SC-8923), actin antibody (Chemicon MAB1501), tubulin antibody (Chemicon MAB380), and NMDAR antibody (Santa cruz (C-29) SC-1467) were used. The results showed that PSD-95 and Homerl b/c were contained in soluble fractions 17 to 24, which were then collected and separated by 1D-PAGE using a 10% acrylamide gel. The PAGE gel was excised in 1 to 2 millimeter width, and trypsin digested. After that, identification by mass spectrometry was performed using LCQ (classic) (Thermo Finnigan Inc, San Jose, Calif.). The molecules contained in fractions 17 to 24 are shown in Table 1.

TABLE 1

| Types | Proteins Identified |
|---|---|
| Scaffold protein | PSD-95 |
| | Homer 1b |
| | SAPAP |
| | BEGAIN |
| | WAVE |
| Transmembrane protein | Densin-180 |
| | Ca channel |
| | T-Cadherin |
| | Thy-1 |
| Signal transduction protein | CaM kinase II |
| Cytoskeletal protein | Actin |
| | Actinin |
| | Tubulin |
| | Spectrin |
| | Cortactin binding protein1 |
| | Nerofilament-H, -M, -L |
| | Neurabin 1, 2 |
| Presynaptic membrane | Rim |
| protein | |
| Others | Ribosomal protein |
| Unknown | EST clone (MS733) |

### Example 4

### Homology Search for MS733

A functionally unknown EST clone (MS733) (IMAGE consortium) identified in Example 3 was used as a probe to clone the corresponding full-length sequence. The nucleotide sequence of the full-length MS733 sequence was determined (SEQ ID NO: 2) and a database search (GenBank; blast search) was performed.

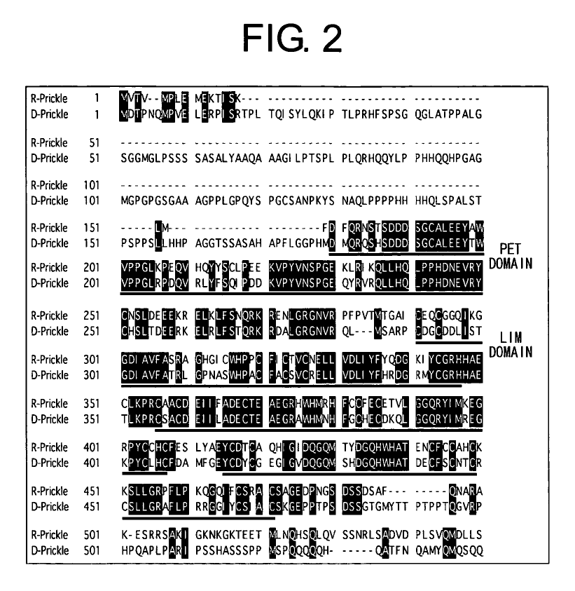

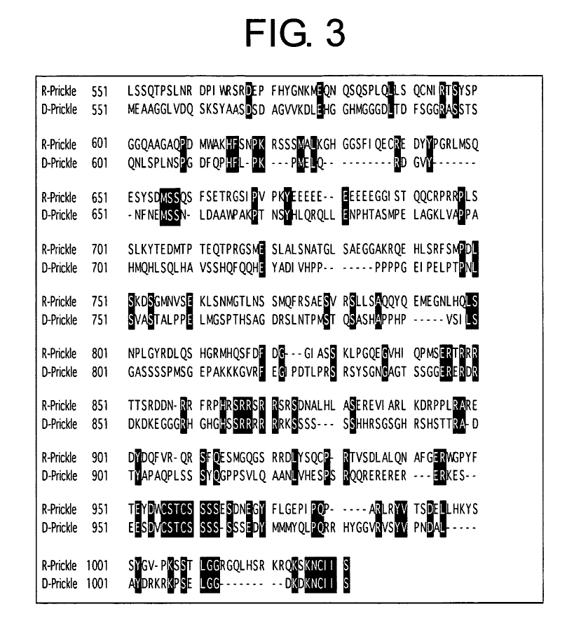

The results showed an approximately 23% homology of MS733 with D-Prickle. Accordingly, the present MS733 protein was named Rat Prickle (R-Prickle). Comparison of D-Prickle and R-Prickle at the amino acid sequence level is shown in FIG. 2. The amino acid sequences of R-Prickle and D-Prickle are shown as SEQ ID NOs: 1 and 3, respectively. R-Prickle of the present invention consists of 847 amino acid residues, and has one PET domain in the N terminus, and three LIM domains (FIGS. 2 and 3). This structure is well conserved in the Prickle family (FIG. 3).

### Example 5

### Histological Distribution of R-Prickle

Homogenates of rat tissues (20 μg protein) were subjected to SDS-PAGE and Western blotting with an anti-prickle antibody.

First, female rats (Japan SLC Inc.) were anesthetized with diethyl ether, and perfused with PBS buffer. After that, each organ was removed, and homogenized using a Teflon® homogenizer, following the addition of about ten volumes of PBS buffer. Homogenates were centrifuged at 800×g for ten minutes, and the resultant supernatants were used as tissue homogenates.

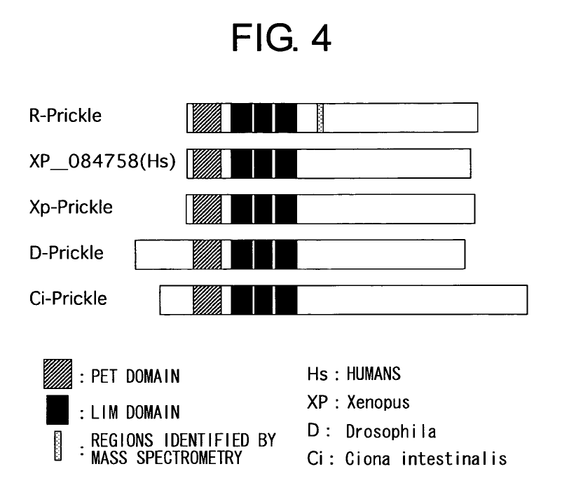

Next, the mPrickle portion consisting of amino acid residues 365 to 618 was expressed as a GST fusion protein for preparation of rabbit antiserum. The antiserum was affinity-purified, and used as an anti-Prickle antibody for Western blotting. See, the Ohtsuka et al method (Ohtsuka et al. (2002) “Cast: a novel protein of the cytomatrix at the active zone of synapses that forms a ternary complex with RIM1 and munc 13-1.” J. Cell Biol. 158: 577-90), for detailed procedures for antibody preparation. As a result, two bands were observed in the brain sample (FIG. 4). With prolonged exposure, a signal was also detected in the skeletal muscle sample. This suggests that R-Prickle is strongly expressed in the brain.

### Example 6

### Intracellular Distribution of R-Prickle in Rat Brain

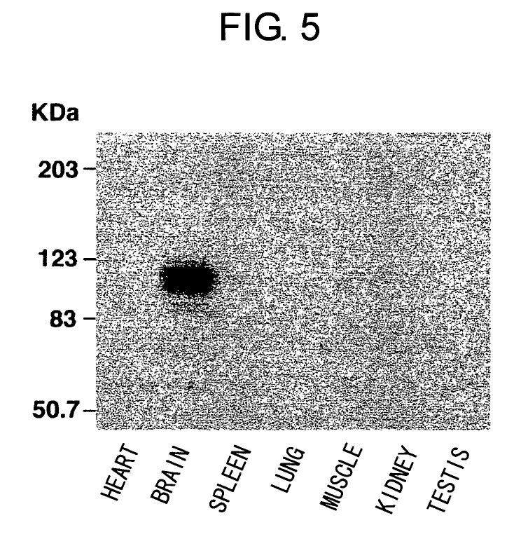

Subcellular fraction (10 μg protein) was subjected to SDS-PAGE, and Western blotting with the anti R-Prickle antibody. The subcellular fraction was prepared according to the Ohtsuka et al. method (Ohtsuka et al. (2002) “Cast: a novel protein of the cytomatrix at the active zone of synapses that forms a ternary complex with RIM1 and munc 13-1.” J. Cell Biol. 158: 577-90). The results showed that R-Prickle was highly concentrated in the PSD fraction, as is the case of NMDA receptors (FIG. 5).

### Example 7

### R-Prickle Solubilization

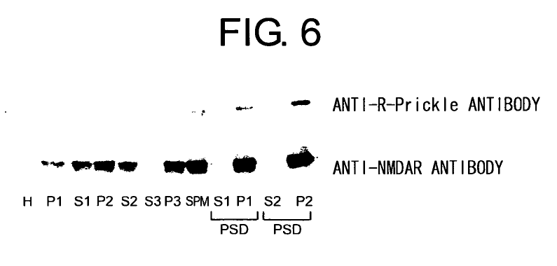

Next, solubilization of R-Prickle was carried out using amphoteric (1% CHAPS [Nacalai Tesque]), non-ionic (1% NP40 [Nacalai Tesque], 1% Triton-X100 [Nacalai Tesque]), and ionic (1% SDS [Nacalai Tesque], 1% DOC [Nacalai Tesque]) surfactants. R-Prickle was prepared according to the Ohtsuka et al. method (Ohtsuka et al. (2002) “Cast: a novel protein of the cytomatrix at the active zone of synapses that forms a ternary complex with RIM1 and munc 13-1.” J. Cell Biol. 158: 577-90). The results showed that while R-Prickle was hardly solubilized in CHAPS, NP-40, and TritonX-100, it was partially solubilized in DOC, and almost completely solubilized in SDS (FIG. 6). Accordingly, R-Prickle was suggested to bind strongly to the cytoskeleton in synaptic junctions.

### Example 8

### R-Prickle Expression at each Developmental Stage

Brain homogenates of R-Prickle rats at developmental stages from embryonic (E18) to postnatal (P70) were applied to SDS-PAGE.

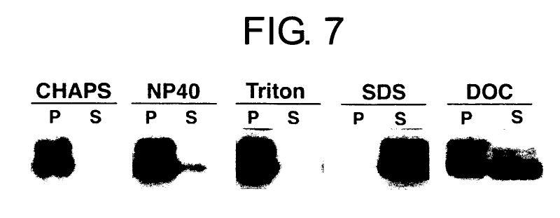

The brain homogenates were prepared from rats (Japan SLC Inc.) according to the method described in Example 5. Western blotting was then performed using the anti R-Prickle antibody. The results revealed that the R-Prickle expression reached its peak at P14 (FIG. 7).

### Example 9

### R-Prickle Localization in Primary Cultures of Neurons

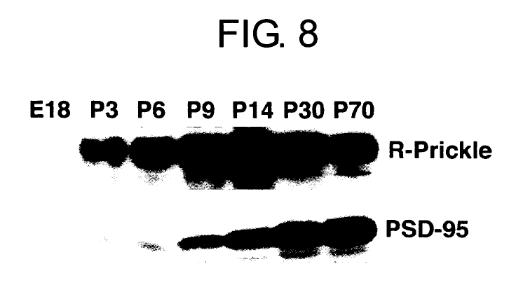

R-Prickle localization in primary cultures of rat embryo hippocampal neurons was examined using the anti R-Prickle antibody. Cells cultured for 28 days were fixed and co-stained with synaptophysin (a presynaptic membrane marker), bassoon (an active zone marker), and PSD-95 (a postsynaptic membrane marker) (Ohtsuka et al. (2002) “Cast: a novel protein of the cytomatrix at the active zone of synapses that forms a ternary complex with RIM1 and munc 13-1.” J. Cell Biol. 158: 577-90) (FIG. 8). The localization of R-Prickle closely matched with those of the markers, and was consistent with that of PSD-95 in particular, suggesting that R-Prickle is localized to postsynaptic membranes.

### INDUSTRIAL APPLICABILITY

The present invention provides the mPrickle gene, a gene encoding the protein that has been known as MS733 and that is present in PSD fractions. The mPrickle protein, which was confirmed to be located at synapses by the present invention, can be used as a synaptic marker. In addition, the mPrickle protein was shown to bind to PSD-95, which is known to be a scaffold protein, and thus it can also be applied to PSD-95 purification. Furthermore, NMDA receptors are co-precipitated when an antibody against mPrickle is used to precipitate endogenous mPrickle. Thus, the mPrickle protein can be used in drug delivery systems that target NMDA receptors. Moreover, NMDA receptors are closely related to learning and memory, and their involvement in mental diseases is also suggested. Accordingly, the use of mPrickle protein or gene, whose relationship with the NMDA receptors has been elucidated, in studying the expressions and functions of NMDA receptors at synapses is considered important for understanding memory and learning, stages of neural development, and mechanisms of neurodegenerative diseases. In the future, mPrickle is expected to be applicable to the diagnosis and/or treatment of diseases associated with learning and memory, such as mental deterioration and dementia.

1. A polynucleotide encoding mammalian Prickle protein, wherein the polynucleotide comprises a sequence selected from the following nucleic acid sequences of (1) to (4):
(1) a nucleic acid sequence that encodes the amino acid sequence of SEQ ID NO: 1, or a complementary sequence thereof;
(2) the nucleic acid sequence of SEQ ID NO: 2, or a complementary sequence thereof;
(3) a nucleic acid sequence that encodes an amino acid sequence with one or more amino acid deletions, insertions, substitutions, or additions to the amino acid sequence of SEQ ID NO: 1, or a sequence complementary to said nucleic acid sequence; and
(4) a nucleic acid sequence that hybridizes with the sequence of (2) under stringent conditions.

2. A vector comprising the polynucleotide of claim 1.

3. A host cell comprising the polynucleotide of claim 1.

4. A method for producing a mammalian Prickle protein encoded by the polynucleotide of claim 1, wherein the method comprises the step of translating said polynucleotide.

5. A fragment of a polypeptide encoded by the polynucleotide of claim 1, wherein the fragment comprises at least eight amino acid residues.

6. An antibody directed against a polypeptide encoded by the polynucleotide of claim 1.

7. A nucleotide chain that encodes the polypeptide fragment of claim 5.

8. A host cell comprising the vector of claim 2.

9. An antibody directed against the polypeptide fragment of claim 5.


SEQUENCE LISTING (3 sequences)

SEQ ID NO: 1 - protein, 847 aa, Rattus norvegicus
MVTVMPLEMEKTISKLMFDFQRNSTSDDDSGCALEEYAWVPPGLKPEQVHQYYSCLPEEKVPYVNSPGEKLRIKQLLHQLPPHDNEVRYCNSLDEEEKRELKLFSNQRKRENLGRGNVRPFPVTMTGAICEQCGGQIKGGDIAVFASRAGHGICWHPPCFICTVCNELLVDLIYFYQDGKIYCGRHHAECLKPRCAACDEIIFADECTEAEGRHWHMRHFCCFECETVLGGQRYIMKEGRPYCCHCFESLYAEYCDTCAQHIGIDQGQMTYDGQHWHATENCFCCAHCKKSLLGRPFLPKQGQIFCSRACSAGEDPNGSDSSDSAFQNARAKESRRSAKIGKNKGKTEETMLNQHSQLQVSSNRLSADVDPLSVQMDLLSLSSQTPSLNRDPIWRSRDEPFHYGNKMEQNQSQSPLQLLSQCNIRTSYSPGGQAAGAQPDMWAKHFSNPKRSSSMALKGHGGSFIQECREDYYPGRLMSQESYSDMSSQSFSETRGSIPVPKYEEEEEEEEEEGGISTQQCRPRRPLSSLKYTEDMTPTEQTPRGSMESLALSNATGLSAEGGAKRQEHLSRFSMPDLSKDSGMNVSEKLSNMGTLNSSMQFRSAESVRSLLSAQQYQEMEGNLHQLSNPLGYRDLQSHGRMHQSFDFDGGIASSKLPGQEGVHIQPMSERTRRRTTSRDDNRRFRPHRSRRSRRSRSDNALHLASEREVIARLKDRPPLRAREDYDQFVRQRSFQESMGQGSRRDLYSQCPRTVSDLALQNAFGERWGPYFTEYDWCSTCSSSSESDNEGYFLGEPIPQPARLRYVTSDELLHKYSSYGVPKSSTLGGRGQLHSRKRQKSKNCIIS

SEQ ID NO: 2 - DNA, 3385 bp, Rattus norvegicus
  CDS (403)...(2943)
gaattcggcacgaggtttagaggagaagggttcttgagaagacaattccaggactcaggcaggcctagaacaagggcacaagtcgcactctctggcttggtggatctctagcagggtctgctttcagggagggcgttctctcggagggaatgcaaaagaagaccggtttgggtttgagcctgctgcctgcctccggaagggctcagattcgcagagactctgtaaaggagatgtttgggctgcctggggtcctgcttgcattttctggtaccagctccagggggaaagtgaactggggcaactgaccagatcaagactcggtggcggcttcttctaaagcttcgcggctcatcctctaggaggaagatctgcctgcactgcaagtgtccccaggaggagcacatggtgacagtgatgccgctggagatggagaagaccattagcaagctcatgtttgacttccagaggaattcaacctcagatgacgactcgggctgtgctttggaagaatatgcctgggtccccccggggctgaagcctgagcaggtgcaccagtattatagctgcctcccagaagagaaagttccctatgtcaacagccctggagagaaattgcgaatcaagcagctactgcaccagctgccaccccacgacaatgaggttcggtattgcaactccctggacgaggaggagaagcgggagctgaagctgttcagcaaccagaggaaacgtgagaacctgggccgaggcaatgtcaggcccttccccgtcaccatgacaggggctatttgcgaacagtgtggaggtcagattaagggcggcgacatcgctgtgtttgcatcacgtgctggccatggcatctgctggcatccaccatgctttatatgcacagtctgcaatgagcttctggttgacctgatctacttttaccaagatgggaagatctactgtggcaggcaccatgctgagtgcctgaagccgcgctgtgcagcctgtgatgagatcatctttgcagatgagtgcacagaggctgaaggacgacattggcacatgagacacttctgctgcttcgagtgtgagacggtgctggggggccagcgctacatcatgaaggagggaagaccctactgctgtcactgctttgagtccctgtatgcagaatattgtgacacctgtgcccaacatataggaattgaccaaggtcaaatgacctatgacggccaacactggcatgctacggagaactgtttctgctgtgctcattgtaagaagtctctcctggggcggccattcctcccgaagcaaggccagatattctgctcacgggcctgtagtgctggggaggaccccaatggctctgactcatctgattcagccttccagaatgccagagccaaggagtctcgccgcagtgccaaaattggcaagaacaagggcaaaacagaggaaaccatgctgaaccagcacagccagctgcaggtgagttctaaccggctctcagctgatgtggaccccctgtcagtgcagatggatctcctcagcctgtccagccagacgcccagcctcaaccgggaccccatttggaggagccgggatgagcccttccattacgggaacaagatggagcagaaccaatcccagagtcctttgcagctcctcagccagtgcaacatcagaacgtcctacagtccaggagggcaggcagcaggagcccagcctgacatgtgggccaagcacttcagcaaccccaagagaagctcatcaatggccctgaaggggcacggtggcagttttatccaggaatgtcgtgaggactattacccagggaggctgatgtcccaggagagctacagcgatatgtctagccaaagctttagtgaaacccgaggcagcatcccggttcccaagtatgaggaggaggaggaggaggaggaggaagaaggtggcatatctacccagcagtgccggccccggcgtccactcagctccctgaaatacacagaggacatgacacccacagaacagactcctcgtggctctatggaatcactggccctgtcaaatgcaacaggcctgtctgctgaaggtggtgccaagcgccaggagcacctatcccggttttccatgcctgacctcagcaaagactctggaatgaatgtctcggaaaagctgagcaacatgggcactctcaactcctcaatgcaattcaggagcgcagagtctgtccgcagcctactgtctgcgcaacagtaccaggagatggagggaaaccttcaccagctcagcaaccccctggggtacagggacctacagtcccatggaaggatgcaccagagctttgattttgatggcggcatagcgagcagcaagctaccagggcaggaaggtgttcacatccagcccatgagtgagcgcacccggaggagaactacttccagggatgacaaccgccgcttccgccctcaccggtccaggaggtcccgacgctcacgttcagacaacgccctgcacctagccagcgagcgggaagtcatcgctcggttgaaggataggccccctctgagagccagggaggactatgaccagttcgtgcgccaacgcagttttcaggagagcatggggcagggttcccggcgggatctgtacagccagtgtcccaggactgtgtcagacctggctttgcagaatgcctttggggaacgatggggaccctacttcacggagtatgattggtgctccacctgctcctcctcttcagagtccgacaatgagggctacttcctaggagaacccatcccccagccagcccgcctacgatatgtcacaagtgatgagctcctgcacaagtacagctcttacggtgtccccaaatcctccaccttaggtggcagaggacagttgcacagcaggaaaagacagaagagcaaaaactgtatcatttcgtaatgtgatggtggttggggaatcggggagatgggagctaagaatgtaaactcagaaacgtgcactgttgtgaattctcaagtgctcttggggatggttcatggaggagagatggagaatgctctcagttgagggaggcaaggctgcaggttgacttggtagatacacttggtgtcacagtttgggacgtgttaaatatgatttgcacatttcactttggaaacacaggctgtggaggccagggtaggtcatgtcctttccaaggtggagtggccattaaccagatgacacactgtaacctgctcacttatattctttctggttctcttccttttaggatcccttttccagtaaatactttattagccaggaccccagactaagttatttacatgttcattgtaaatgcaatgtaaatgagagttctgataaaatatttttg

SEQ ID NO: 3 - protein, 963 aa, Drosophila melanogaster
MDTPNQMPVELERPISRTPLTQISYLQKIPTLPRHFSPSGQGLATPPALGSGGMGLPSSSSASALYAAQAAAGILPTSPLPLQRHQQYLPPHHQQHPGAGMGPGPGSGAAAGPPLGPQYSPGCSANPKYSNAQLPPPPHHHHQLSPALSTPSPPSLLHHPAGGTSSASAHAPFLGGPHMDMQRQSHSDDDSGCALEEYTWVPPGLRPDQVRLYFSQIPDDKVPYVNSPGEQYRVRQLLHQLPPHDNEVRYCHSLTDEERKELRLFSTQRKRDALGRGNVRQLMSARPCDGCDDLISTGDIAVFATRLGPNASWHPACFACSVCRELLVDLIYFHRDGRMYCGRHHAETLKPRCSACDEIILADECTEAEGRAWHMNHFGCHECDKQLGGQRYIMREGKPYCLHCFDAMFGEYCDYCGEGIGVDQGQMSHDGQHWHATDECFSCNTCRCSLLGRAFLPRRGGIYCSIACSKGEPPTPSDSSGTGMYTTPTPPTQGVRPHPQAPLPARIPSSHASSSPPMSPQQQQQHQATFNQAMYQMQSQQMEAAGGLVDQSKSYAASDSDAGVVKDLEHGGHMGGGDLTDFSGGRASSTSQNLSPLNSPGDFQPHFLPKPMELQRDGVYNFNEMSSNLDAAWPAKPTNSYHLQRQLLENPHTASMPELAGKLVAPPAHMQHLSQLHAVSSHQFQQHEYADIVHPPPPPPGEIPELPTPNLSVASTALPPELMGSPTHSAGDRSLNTPMSTQSASHAPPHPVSILSGASSSSPMSGEPAKKKGVRFEGIPDTLPRSRSYSGNGAGTSSGGERERDRDKDKEGGGRHGHGHSSRRRRRRKSSSSSSHHRSGSGHRSHSTTRADTYAPAQPLSSSYQGPPSVLQAANLVHESPSRQQRERERERERKESEESDVCSTCSSSSSSSEDYMMMYQLPQRRHYGGVRVSYVPNDALAYDRKRKPSELGGDKDKNCIIS